(19) United States
(12) Patent Application Publication
(10) Pub. No.: US 2018/0092800 A1
(43) Pub. Date: Apr. 5, 2018

(54) THERAPEUTIC MASSAGE MAT

(71) Applicant: Lawrence D. Chapman, Littleton, CO (US)

(72) Inventor: Lawrence D. Chapman, Littleton, CO (US)

(21) Appl. No.: 15/352,026

(22) Filed: Nov. 15, 2016

Related U.S. Application Data
(60) Provisional application No. 62/404,499, filed on Oct. 5, 2016.

(51) Int. Cl. A61H 23/00; A61N 2/00; A61N 5/06

(52) U.S. Cl. CPC A61H 23/00; A61N 2/002; A61N 2005/067; A61H 2203/0468; A61N 5/0613

(57) ABSTRACT

A therapeutic massage mat is provided with one or more healing agents. The therapeutic massage mat includes: (i) a top layer, (ii) a middle layer, (iii) a bottom layer, (iv) an array of layered sheets, (v) one or more bags, (vi) one or more motors, and (vii) a Velcro. The top layer is adapted to allow one or more healing agents to pass through. The array of layered sheets is coupled to the top layer. The one or more bags are adapted to carry the one or more healing agents. The one or more bags are coupled to one or more laser diodes. The one or more healing agent are yellow tourmaline powder, magnetite, silicon infused crystal powder, and FIR pad. The Velcro is coupled to the top layer, the middle layer and the bottom layer.

### BACKGROUND

### Technical Field

This application claims priority to and the benefits of the provisional patent application No. “62/404,499” titled “THERAPEUTIC MASSAGE MAT” filed in United States Patent and Trademark Office on “Oct. 05, 2016”.

The present invention generally relates to a therapeutic massage mat and more particularly to a functional health massage mat with one or more health benefits.

### Description of the Related Art

People in modern world live a busy life. Stressful lifestyles and lack of exercise make people unhealthy and prone to several ailments like obesity and allergies. People with chronic pain lack sufficient energy to get adequate relief. This invention supplies many healing energies that provide the mind and body with a way to alleviate or cure the cause of this chronic pain. For example Negative Ions, Magnetic energy, several different crystal energies, Far-infrared and pulsed laser energy reaching deep into the flesh

Massage is a well known form of body work and physical therapy. Body massage may also be quite helpful for the body healing and relaxation. Body massage pads and foot mats work on the body of a user including, without limiting, back, shoulder, neck, abdomen, feet, hips and/or head. The body massage pads help to eliminate minor muscle aches, pain, stress and/or tension. The massage may take some time for the healing of the pain. The addition of the healing energies provides immediate relief and speeds the healing by many times.

Accordingly, there remains a need for a simple and inexpensive massage mat with healing agents.

### SUMMARY

In view of the foregoing, an embodiment herein provides a therapeutic massage mat with one or more healing agents. The therapeutic massage mat includes: (i) a top layer, (ii) a middle layer, (iii) a bottom layer, (iv) an array of layered sheets, (v) one or more bags, (vi) one or more motors, and (vii) a Velcro. The top layer is adapted to allow one or more healing agents to pass through. The array of layered sheets is coupled to the top layer. The one or more bags are adapted to carry the one or more healing agents. The one or more bags are coupled to one or more laser diodes.

In one embodiment, the Velcro is coupled to the top layer, the middle layer, and the bottom layer. The one or more healing agents are selected from a group of tourmaline powder, magnetite, crystal powder and FIR pad. The tourmaline powder is a yellow tourmaline powder. The crystal power is selected from a group of clear crystals, rose crystals, citrine crystals, Lapis Lazuli, and amethyst crystals. The crystal powder is infused in silicone. The one or more bags are one or more stiff vinyl bags. The one or more bags extend across the entire length of the therapeutic massage mat. In another embodiment, the yellow tourmaline ranges from 70-80%. The composition of the magnetic powder ranges from 5-15%. The composition of the silicone ranges from 5-15%.

In yet another aspect of the present invention, a therapeutic massage mat is provided with one or more healing agents. The therapeutic massage mat includes: (i) a top layer, (ii) a middle layer, (iii) a bottom layer, (iv) an array of layered sheets, (v) one or more bags, (vi) one or more motors, and (vii) a Velcro. The top layer is adapted to allow one or more healing agents to pass through. The array of layered sheets is coupled to the top layer. The one or more bags are adapted to carry the one or more healing agents. The one or more bags are coupled to one or more laser diodes. The one or more healing agent are yellow tourmaline powder, magnetite, silicon infused crystal powder, and FIR pad. The Velcro is coupled to the top layer, the middle layer and the bottom layer. The crystal power is selected from a group of clear crystals, rose crystals, citrine crystals, Lapis Lazuli, and amethyst crystals.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a perspective view of a therapeutic massage mat according to an embodiment herein;

FIG. 2 illustrates an exploded view of the therapeutic massage mat of FIG. 1 according to an embodiment herein; and

FIG. 3 is a diagrammatic representation of one or more laser diodes arranged around one or more bags according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

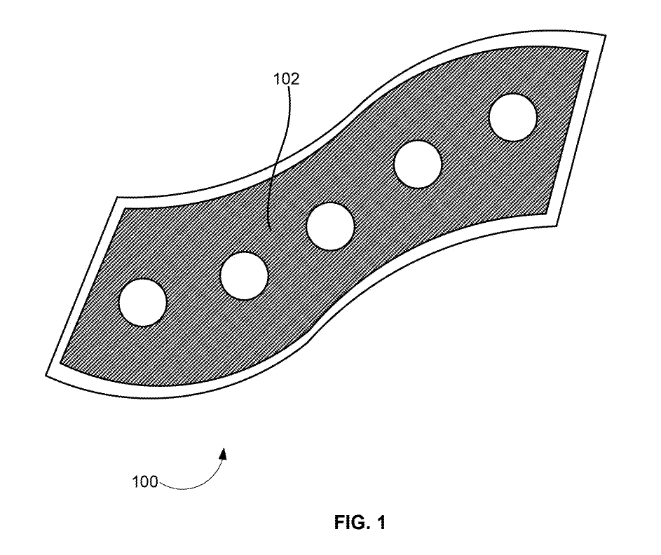

As mentioned, there remains a need for a simple and inexpensive massage mat with healing agents. The embodiment herein achieves this by providing a therapeutic massage mat with one or more healing agents. Referring now to the drawings, and more particularly to FIGS. 1 through 3, where similar reference characters denote corresponding features consistently throughout the figures, there are shown preferred embodiments.

FIG. 1 is a perspective view of a therapeutic massage mat 100 with healing agents according to an embodiment herein. The healing agents enhance the repair of torn tissues or the like. In one embodiment, the healing agents may be, without limiting, tourmaline powder, magnetite, crystal powder, laser diodes, FIR pad. The therapeutic massage mat 100 massages a user and the healing agents are to heal the user's problems. In one embodiment, the therapeutic massage mat 100 is operated by electric power.

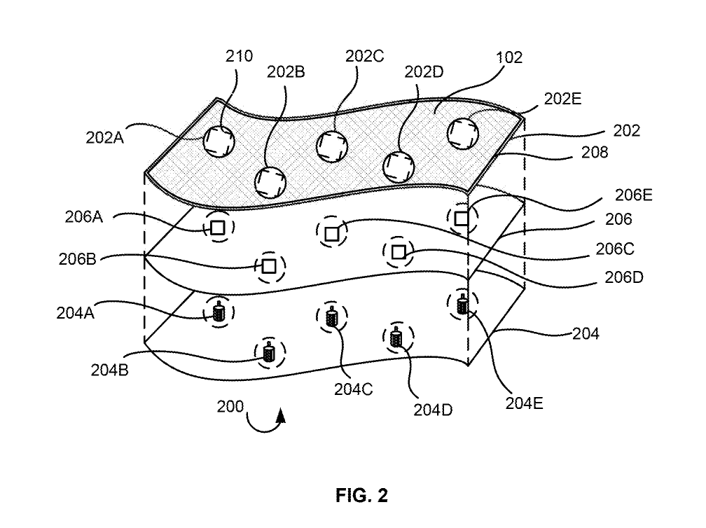

FIG. 2 illustrates an exploded view 200 of the therapeutic massage mat 100 of FIG. 1 according to an embodiment herein. The therapeutic massage mat 100 includes a FIR pad 102, top layer 202, an array of layered sheets 202A-202E, a bottom layer 204, a first motor 204A, a second motor 204B, a third motor 204C, a fourth motor 204D, a fifth motor 204E, a middle layer 206, a first bag 206A, a second bag 206B, a third bag 206C, a fourth bag 206D, a fifth bag 206E, and a Velcro 208.

The array of layered sheets 202A-202E is coupled to the top layer 202 to allow one or more materials to pass through. The five motors 204A-204E are coupled to the bottom layer 204. The five motors 204A-204E provides vibrations which help in relaxing the aches and fixing the torn tissues of a user. In one embodiment, the five motors 204A-204E are attached throughout the length of the therapeutic massage mat 100. In another embodiment, the five motors 204A-204E are coated with yellow tourmaline (preferably 70-80%), magnetic powder (preferably 5-15%) and silicon mixture (preferably 5-15%) may produce more energy. The silicon mixture coating the five motors 204A-204E eliminates the need for bags, springs and a lot of weight. The middle layer 206 rests in between the top layer 202 and the bottom layer 204. The middle layer 206 possesses a provision to fix the first bag 206A, the second bag 206B, the third bag 206C, the fourth bag 206D and the fifth bag 206E. In one embodiment, the first bag 206A, the second bag 206B, the third bag 206C, the fourth bag 206D and the fifth bag 206E are stiff vinyl bags and extend across the entire length of the massage mat. In another embodiment, a set of 5 bags (not shown in figure) may be filled with equally divided crystal powders. The bags rest upon the bags containing the yellow tourmaline powder and magnetic powder mixture.

The five bags 206A-206E of the therapeutic massage mat 100 are filled with yellow tourmaline powder and magnetic powder mixture. When the therapeutic massage mat 100 is started, the five motors 204A-204E runs and vibrates the tourmaline powder and magnetic powder mixture inside the five bags 206A-206E. In one embodiment, the therapeutic massage mat 100 may employ 5-20 motors, more preferably 10 motors, throughout the length of the therapeutic massage mat 100, as increase in number of motors increases the vibrations. The five bags 206A-206E are in contact with the array of layered sheets 202A-202E where the user is resting. The vibrations of the five motors 204A-204E force the energy of the tourmaline powder and magnetic powder to penetrate out of the array of perforated sheets 202A-202E, so the user gets massaged with the vibration of the massage motors together with the energy of the negative ions from the cloth covering as well as the FIR radiation. In one embodiment, the speed of the five motors 204A-204E may be varied using a regulator.

The therapeutic massage mat 100 utilizes naturally existing negative ion element like tourmaline. The tourmaline is a well known gemstone for an ability to aid the detoxification process of the user's body. The tourmaline stone is in solid and/or powder form that emits negative ions. In another embodiment, the tourmaline may become a source of electric charge. The user massages the affected area with the tourmaline powder to get healed quickly.

The magnetite/magnetic powder help to energize and boost the circulation rate of blood and enhance metabolic activities of the user. The Magnetite further stimulates nutrient rich blood flow thereby helping organs and tissues of the user's body in getting oxygenated blood. Scientifically, the magnetite helps in excretion of more endorphins that are user's body's natural pain killers and hence promote repair and faster healing. The magnetic fields help to reduce inflammation and body stiffness and cell degeneration. When the user uses the therapeutic massage mat 100 on head, the user feels relieved from stress. The user massages the affected area with the magnetic powder to get healed quickly.

The set of 5 bags (not shown in figure) of the therapeutic massage mat 100 is filled with one or more crystals. In one embodiment, the one or more crystals may be, without limiting, clear crystals, rose crystals, citrine crystals, Lapis Lazuli, amethyst crystals or the like, in the form of sand or powder. When the therapeutic massage mat 100 is started, the five motors 204A-204E run and vibrate the crystal powder inside the set of 5 bags. The set of 5 bags is in contact with the array of perforated sheets 202A-202E where the user is resting. The vibrations of the five motors 204A-204E force the energy of the crystal powder to penetrate out of the array of perforated sheets 202A-202E, so the user gets the energy with the massage vibrations. In one embodiment, the crystal powder is infused in silicone. In another embodiment, the silicone infused crystal powder is mixed with yellow tourmaline powder and magnetic powder mixture.

The one or more crystals possess several health benefits, non limiting examples are, (i) rose quartz cures heartaches; (ii) amethyst crystal brings strength, peace and courage to the user. The amethyst promotes healing and cures anxiety, mood disorders and addictions. Further, the citrine crystal helps promote emotional well being and positiveness of the user. The citrine crystal helps to overcome sleep problems, pain and inflammation management and also provides additional needed energy or the like. Lapis lazuli is known for relief of pain. The user massages the affected area with the crystal powder to get healed quickly.

The five bags 206A-206E of the therapeutic massage mat 100 are coupled with one or more laser diodes. The five boxes 206A-206E are in contact with the array of layered sheets 202A-202E where the user is resting. The non-invasive light penetrates out of the array of perforated sheets 202A-202E and heals the user. The laser diodes are used for low level laser therapy (LLLT) The one or more laser diodes emit non-invasive light. The non-invasive light is beneficial for injured, inflamed, or diseased tissue. Scientifically, it is known that photons of light stimulate ATP production and accelerate the healing process. In one embodiment, LLT cures sports related injuries, stress, inflammatory conditions and back related problems.

The top cover of the therapeutic massage mat 100 holds the Far Infrared Ray (FIR) Pad 102 being infused with Silicone mixture. The FIR Cloth is also a source of high negative ion emission. The top cover 206E is in contact with the array of layered sheets 202A-202E where the user is resting. The FIR penetrates out of the array of perforated sheets 202A-202E and heals the user. The therapeutic massage mat 100 further uses benefits of Far Infrared Rays Therapy (FIR). The Far Infrared Rays gently heal, soothe and stimulate the body and mind of the user. The FIR penetrates the layers of the human body and thus enters the innermost bone, muscles and/or tissues. The FIR elevates the body's surface temperature slightly and helps in enhanced body functionality at multiple levels. The user applies the FIR area of the therapeutic massage mat 100 to the affected part of the user's body and finds the cure to the ailment. The top cover of the therapeutic massage mat 100 further includes one or more yellow tourmaline infused decorative patches 210. In one embodiment, the one or more yellow tourmaline infused decorative patches are bonded with the therapeutic massage mat 100 using glue. In another embodiment, the infused yellow silicone and cloth top of the therapeutic massage mat 100 may produce a large number of negative ions and more far-infrared energy.

In another embodiment, the FIR possesses the benefits in fat loss, skin diseases, water retention, fatigue handling elimination of carcinogens and heavy metal from body or the like. Furthermore, the FIR slightly expands capillaries which increase blood flow. The FIR also increases oxygenation and regeneration of the blood and helps in detoxification at the deepest level.

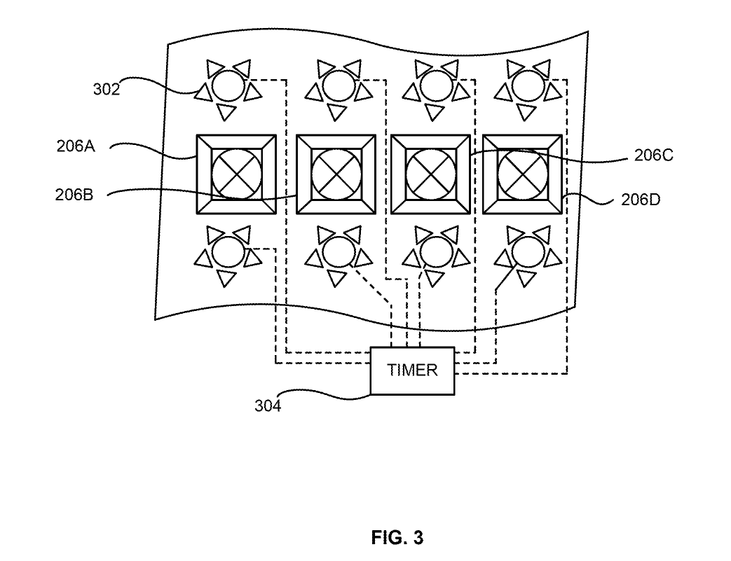

FIG. 3 is a diagrammatic representation of one or more laser diodes 302 arranged around one or more bags 206A-206E according to an embodiment herein. The sides of the one or more bags 206A-206E accommodate one or more hard rubbers (not shown in FIG. 3). Each hard rubber possesses a hole in the middle for the insertion of the one or more laser diodes 302. In one embodiment, the one or more hard rubbers may possess any shape, including without limiting, circular, oval, square or rectangular. One or more laser diodes 302 point towards the body of the user. A timer 304 connects all of more laser diodes 302. The timer 304 allows the laser to penetrate for a limited time to allow safe amount of laser energy to radiate the body of the user.

In another embodiment, the first bag 206A, the second bag 206B, the third bag 206C, the fourth bag 206D and the fifth bag 206E may be replaceable and/or refillable as per the need of the user. In one embodiment, the therapeutic massage mat 100 of may be disassembled in order to refill the first bag 206A, the second bag 206B, the third bag 206C, the fourth bag 206D and/or the fifth bag 206E. A Velcro 208 provides the flexibility to disassemble all the layers of the therapeutic massage mat 100 from each other. Furthermore, the top layer 202, the bottom layer 204 and the middle layer 206 are separable from each other in case of any repair.

In one embodiment, the therapeutic massage mat 100 may be attached to an easy chair placed in malls, offices or residences to relieve the pain of the user. In another embodiment, the therapeutic massage mat 100 may be in the form of a bed (preferably, a hospital bed) to relieve the pain. In yet another embodiment, the therapeutic massage mat 100 may additionally include hand held massagers, gloves for massage therapists, and a helmet. The helmet puts energy into one or more acupuncture points or meridians in the head of the user. In another embodiment potable massagers may be attached to the body, with a rechargeable battery pack to wear during the time the person has to work or be away from the massage mat.

In another embodiment, the five bags 206A-206E are coupled with one or more springs (not shown in FIG. 2) on the base of the five bags 206A-206E. The springs facilitate maximum amount of shaking of the material present in the five bags 206A-206E.

In yet another embodiment, the mode of therapeutic massage with healing agents may be, without limiting, vibration, undulation, pulsing, oscillation, and any other type of movement, along with mechanical adjustments.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A therapeutic massage mat with one or more healing agents, wherein said therapeutic massage mat comprises:
a top layer that is adapted to allow one or more healing agents to pass through;
a middle layer;
a bottom layer;
an array of layered sheets, wherein said array of layered sheets is coupled to said top layer;
one or more bags that are adapted to carry said one or more healing agents, wherein said one or more bags are coupled to one or more laser diodes;
one or more motors; and
a Velcro.

2. The therapeutic massage mat of claim 1, wherein said Velcro is coupled to said top layer, said middle layer and said bottom layer.

3. The therapeutic massage mat of claim 1, wherein said one or more healing agents are selected from a group of tourmaline powder, magnetite, crystal powder and FIR pad.

4. The therapeutic massage mat of claim 3, wherein said tourmaline powder is a yellow tourmaline powder.

5. The therapeutic massage mat of claim 3, wherein said crystal power is selected from a group of clear crystals, rose crystals, citrine crystals, Lapis Lazuli, and amethyst crystals.

6. The therapeutic massage mat of claim 3, wherein said crystal powder is infused in silicone.

7. The therapeutic massage mat of claim 1, wherein said one or more bags are one or more stiff vinyl bags.

8. The therapeutic massage mat of claim 1, wherein said one or more bags extend across the entire length of said therapeutic massage mat.

9. The therapeutic massage mat of claim 4, wherein composition of said yellow tourmaline ranges from 70-80%.

10. The therapeutic massage mat of claim 3, wherein composition of said magnetic powder ranges from 5-15%.

11. The therapeutic massage mat of claim 6, wherein composition of said silicone ranges from 5-15%.

12. A therapeutic massage mat with one or more healing agents, wherein said therapeutic massage mat comprises:
a top layer that is adapted to allow one or more healing agents to pass through;
a middle layer;
a bottom layer;
an array of layered sheets, wherein said array of layered sheets is coupled to said top layer,
one or more bags, that are adapted to carry said one or more healing agents, wherein said one or more bags are coupled to one or more laser diodes, wherein said one or more healing agent are yellow tourmaline powder, magnetite, silicon infused crystal powder, and FIR pad;
one or more motors; and
a Velcro, wherein said Velcro is coupled to said top layer, said middle layer and said bottom layer.

13. The therapeutic massage mat of claim 12, wherein said crystal power is selected from a group of clear crystals, rose crystals, citrine crystals, Lapis Lazuli, and amethyst crystals.

14. The therapeutic massage mat of claim 12, wherein composition of said yellow tourmaline ranges from 70-80%.

15. The therapeutic massage mat of claim 12, wherein composition of said magnetic powder ranges from 5-15%.

16. The therapeutic massage mat of claim 12, wherein composition of said silicone ranges from 5-15%.
